(19) United States
(12) Patent Application Publication
(10) Pub. No.: US 2022/0370537 A1
(43) Pub. Date: Nov. 24, 2022

(54) PHARMACEUTICAL COMPOSITION AND FUNCTIONAL HEALTH FOOD FOR TREATING BENIGN PROSTATIC HYPERPLASIA INCLUDING LAURUS NOBILIS AS ACTIVE INGREDIENT, AND METHOD OF TREATING BENIGN PROSTATIC HYPERPLASIA USING THE SAME

(71) Applicant: KOSABIO INC., Namyangju-si (KR)

(72) Inventors: Kyung-Jae KIM, Namyangju-si (KR); Young-Cheon SONG, Seoul (KR); Hyun-Seok KONG, Seoul (KR); Ji-Yeon KIM, Seoul (KR); Do-Hyun CHOI, Pocheon-si (KR); Jin-Ho AN, Seoul (KR)

(73) Assignee: KOSABIO INC., Namyangju-si (KR)

(21) Appl. No.: 17/882,030

(22) Filed: Aug. 5, 2022

Related U.S. Application Data
(63) Continuation of application No. PCT/KR2020/002567, filed on Feb. 21, 2020.

(51) Int. Cl. A61K 36/54; A61K 9/19; A61P 13/08

(52) U.S. Cl. CPC A61K 36/54; A61K 9/19; A61P 13/08

(57) ABSTRACT

The present disclosure relates to a pharmaceutical composition and a health composition including an effective amount of a bay leaf extract as an active ingredient for treatment and prevention of benign prostatic hyperplasia, and a method of treating benign prostatic hyperplasia using the same.

### CROSS-REFERENCE TO RELATED APPLICATIONS

Pursuant to 35 USC 120 and 365(c), this application is a continuation of International Application No. PCT/KR2020/002567 filed on Feb. 21, 2020, in the Korean Intellectual Property Office, the entire disclosures of which is incorporated herein by reference for all purposes.

### BACKGROUND

### 1. Field

The present disclosure relates to a pharmaceutical composition and a health functional food for treatment of benign prostatic hyperplasia including a Laurus nobilis extract as an active ingredient, and a method of treating benign prostatic hyperplasia using the pharmaceutical composition.

### 2. Description of Background

Bay laurel (Laurus nobilis) is native to the Mediterranean coast and is mainly distributed in Gyeongnam and Jeonnam regions in the Korean Peninsula. Bay laurel is an evergreen arboreous tree that grows to around 15 m in height and has a dark gray bark and lush branches and leaves. Bay leaves are alternate phyllotaxy, hard, long oval or lanceolate, dark green with wavy edges, and fragrant when rubbed.

Bay leaves have many medicinal uses, but their most impressive benefits include detoxification of the body, protection against bacterial infections, and aiding in fast wound healing. In addition, the leaves are known to have a preventive effect on diabetes mellitus and specific types of cancer.

Bay leaves are used as a diuretic that stimulates urination and reduces body toxicity. Organic compounds found in laurel leaves also relieve irritable bowel syndrome (IBS) and are very effective in alleviating celiac disease (an inherited allergic disease of the small intestine).

One of the greatest benefits of bay leaves is anti-inflammatory efficacy. Bay leaves contain a unique phytonutrient called parthenolide, which can quickly reduce inflammation and irritation when applied topically to infected areas, such as sore joints or those affected by arthritis. In addition, an effect of preventing cancer is also observed. Containing catechins, linalool, parthenolide, and Eugenol, bay leaves can defend the body from free radicals. In other words, the leaves prevent healthy cells from mutating into cancer cells due to free radicals.

In addition, bay leaves are a natural product that has been used for antibacterial, antifungal, and antioxidant performances. Among ingredients found in bay leaves, sesquiterpene lactone inhibits NO production, exhibiting anti-inflammatory activity and enhancing glutathione S-transferase activity in the liver. Costunolide, which is a sesquiterpene lactone, was reported to repress blood ethanol levels as assayed in mice.

The pathogenesis of benign prostatic hyperplasia, although not yet exactly known, is accounted for by enlarged prostate cells induced by excessive production of dihydrotestosterone (DHT). When abundantly present in blood, testosterone is converted by 5α-reductase to a large amount of DHT which, in turn, binds to androgen receptors (AR) in prostate cells to induce benign prostatic hyperplasia. In addition, men decrease in male hormone production with age. Under this condition, the prostate cells of aged men increase the number of ARs in order to maintain the endocrine balance, allowing DHT to bind to more ARs, whereby benign prostatic hyperplasia is caused. As an endogenous factor, the level of an apoptosis regulator in prostate tissues is reportedly increased upon onset of prostate cancer and benign prostatic hyperplasia. Through such pathological mechanisms, the prostate tissue overgrows. Patients with benign prostatic hyperplasia undergo various clinical urination-related symptoms including urinary obstruction, residual urine, detrusor instability, urinary retention, dysuria, and the like and, in severe cases, may suffer from urinary stones, renal failure, hematuria, and infectious complications.

As the aging population increases, prostatic hyperplasia has a significant impact on the quality of life of the male population. Recently, as economic and social conditions have improved, the seriousness of prostatic hyperplasia is recognized as being important, with the consequent expansion of the market for therapeutics therefor.

Until now, there have been a narrow spectrum of therapies for benign prostatic hyperplasia, including alpha-adrenergic receptor blockers that relieve symptoms like dysuria, 5-alpha reductase inhibitors that lower DHT concentrations, and some complementary and alternative therapies.

Examples of alpha adrenergic receptor blockers include terazosin, doxazosin, alfuzosin, and tamsulosin. As 5-alpha reductase inhibitors, finasteride and dutasteride are used. Despite their therapeutic benefits, these drugs have significant restrictions on their use due to their inherent adverse reactions. For instance, side effects include orthostatic hypertension and cardiovascular abnormalities caused by alpha adrenergic receptor blockers and decreased libido caused by 5-alpha reductase inhibitors. For these reasons, complementary and alternative therapeutic agents have been suggested as an alternative, and saw palmetto is used. However, as these therapeutic drugs also have limitations in their efficacy, the need for new therapeutically effective substances is emerging.

Until the present inventors noticed, nothing was known about the effect of laurel on the treatment of benign prostatic hyperplasia. Leading to the present disclosure, intensive and thorough research conducted by the present inventors into development of new therapeutically effective substances resulted in the finding that a bay leaf extract increases immunity and reduces the weight and volume of the prostate, as assayed by cell experiments and benign prostatic hyperplasia-induced animal experiments.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

An aspect of the present disclosure provides a pharmaceutical composition including an effective amount of a bay leaf extract as an active ingredient for treatment and prevention of benign prostatic hyperplasia.

Another aspect of the present disclosure provides a health functional food including an effective amount of a bay leaf extract as an active ingredient for treatment and prevention of benign prostatic hyperplasia.

Still another aspect of the present disclosure provides a method of treating benign prostatic hyperplasia including: preparing a pharmaceutical composition including an effective amount of a bay leaf extract an active ingredient; and administering the pharmaceutical composition to a subject in need of treating the benign prostatic hyperplasia.

The preparing the pharmaceutical composition may include extracting the bay leaf extract from a bay leaf using an extraction solvent.

The extraction solvent may include an ethanol.

The ethanol may be included in the extraction solvent in an amount of 30 vol % to 70 vol % based on a total weight of the extraction solvent.

The preparing the pharmaceutical composition may further include: filtering the bay leaf extract extracted using the extraction solvent; and lyophilizing and powdering the bay leaf extract into a powder.

The powder may be dissolved in a solvent to prepare the pharmaceutical composition in a solution.

The solvent may be dimethyl sulfoxide, distilled water, or a mixture thereof.

The solution may be administered to the subject in an amount of 10 mg/kg to 50 mg/kg based on a body weight of the subject.

Other features and aspects will be apparent from the following detailed description and the claims.

### BRIEF DESCRIPTION OF DRAWINGS

FIGS. 1a and 1b are HPLC chromatograms of 30% and 70% ethanol extracts of bay leaves.

FIG. 2 shows anti-inflammatory effects of 30% and 70% ethanol extracts of bay leaves as assayed for NO production levels of macrophages.

FIGS. 3a and 3b show levels of inflammatory cytokines (IL-1β and IL-6) according to treatment with 30% and 70% ethanol extracts of bay leaves.

FIGS. 4a, 4b, and 4c show therapeutic effects of the 70% ethanol extract of bay leaves on benign prostatic hyperplasia.

FIGS. 5a, 5b, and 5c show prophylactic effects of the 70% ethanol extract of bay leaves on benign prostatic hyperplasia.

### DETAILED DESCRIPTION

The following detailed description is provided to assist the reader in gaining a comprehensive understanding of the compositions and the methods described herein. However, various changes, modifications, and equivalents of the compositions and the methods described herein will be apparent after an understanding of this disclosure. For example, the sequences of operations described herein are merely examples, and are not limited to those set forth herein, but may be changed as will be apparent after an understanding of this disclosure, with the exception of operations necessarily occurring in a certain order. Also, descriptions of features that are known in the art may be omitted for increased clarity and conciseness.

As used herein, “prevention” refers to any action of inhibiting or delaying the onset of benign prostatic hyperplasia by administrating the composition or food composition according to the present disclosure. In addition, it includes treatment of a subject with remission of the disease for the prophylaxis and the prevention of recurrence.

As used herein, “treatment” refers to any action that improves or beneficially alters the condition, such as alleviating, reducing, or eliminating the onset of benign prostatic hyperplasia by administrating the composition or the food composition according to the present disclosure.

As used herein, “improvement” refers to any action of that improves or beneficially alters benign prostatic hyperplasia by ingestion of the composition or the food composition according to the present disclosure.

As used herein, “composition” means a composition administered for a specific purpose, and for the purposes of the present disclosure, it refers to be administered to prevent or treat benign prostatic hyperplasia.

As used herein, “food composition” has a meaning similar to food for specified health use (FoSHU), and refers to foods with high medical and medicinal effects processed so that the biological regulation function is effectively shown in addition to nutritional supply.

The composition and food composition for preventing or treating benign prostatic hyperplasia according to the present disclosure may include “bay leaf extract.”

As used herein, the term “bay leaf” refers to Laurus nobilis in the present disclosure.

As used herein, the term “effective amount” refers to an amount sufficient to treat benign prostatic hyperplasia at a reasonable benefit/risk ratio applicable to medical treatment and does not cause side effects, and the effective dose level can be determined according to factors including the patient's health status, type of disease, severity of disease, activity of the drug, sensitivity to the drug, method of administration, time of administration, route of administration and rate of excretion, duration of treatment, drugs used in combination or concurrently, and other factor well known in the medical field.

In the food composition according to the present disclosure, the food composition may be prepared in various forms such as capsules, tablets, granules, powders, suspensions or syrups.

The food composition may contain various nutrients, vitamins, minerals (electrolytes), flavors such as synthetic flavors and natural flavors, etc., colorants and fillers (cheese, chocolate etc.), pectic acid and its salts, alginic acid and its salts, organic acids, protective colloid thickeners, pH adjusting agents, stabilizers, preservatives, glycerin, alcohols, carbonating agents used in carbonated drinks, and the like. It may also contain flesh for the production of natural fruit juices, synthetic fruit juices and vegetable drinks. These components may be used independently or in combination.

The food composition may be in the form of any one of dairy products, bread, confectionery, ice cream, beverage, tea, functional water and vitamin complex.

In addition, the food composition may further include a food additive and compliance as a food additive is determined by the standards for the applicable item in accordance with General Regulations and General Test Methods of Korean Food Additives Codex approved by the Ministry of Food and Drug Safety, unless otherwise provided.

Examples of the items published in the above-mentioned “Korean Food Additives Codex” include chemical synthetics such as ketones, glycine, potassium citrate, nicotinic acid, and cinnamic acid and the like, natural additives such as persimmon color, licorice extract, crystalline cellulose, kaoliang color and guar gum and the like, mixed preparations such as L-sodium glutamate preparation, alkaline agents for noodles, preservative formulation and a tar color formulation and the like.

According to one embodiment of the present disclosure, the composition or food composition may be administered intravenously, intraarterially, intraperitoneally, intramuscularly, intrasternally, transdermally, nasally, inhaled, topically, rectally, orally, intraocularlly or intradermally to the subject in the conventional manner.

The preferred dosage of the pharmaceutical composition or the food composition may vary depending on the condition and weight of the subject, the type and extent of the disease, the drug form, the route of administration, and the duration, and may be appropriately selected by those skilled in the art. The pharmaceutical composition or the food composition may be administered once a day or divided into several times, and the scope of the invention is not limited thereto.

In the present disclosure, the ‘subject’ may be a mammal including a human, but it is not limited thereto.

The present disclosure has been drawn from the background and is to provide the pharmaceutical composition, the food composition, and the method that allows a bay leaf extract to be applied to the therapy of benign prostatic hyperplasia, as the extract has been proven to be therapeutically effective for benign prostatic hyperplasia.

As will be stated below, the bay leaf extract is effective for treating or preventing benign prostatic hyperplasia.

### MODE FOR CARRYING OUT THE INVENTION

<1. Preparation of Bay Leaf Extract>

Bay leaves used in this experiment were purchased from Heungil dang.

Two rounds of extraction from 2.5 kg of bay leaves were conducted, and the extracts thus obtained were pooled. As extraction solvents, ethanol 30% and 70% were used. Bay leaves were added 20-fold volumes of the solvent and heated for 48 hours. The extracts were obtained at yields of 11.8% and 13.0%, respectively. Each of the extracts were filtered, lyophilized, powdered, and stored at −20° C. until use. In this experiment, the extracts were dissolved at various concentrations in DMSO (1%) or distilled water (D.W).

<2. HPLC Analysis of Bay Leaf Extract>

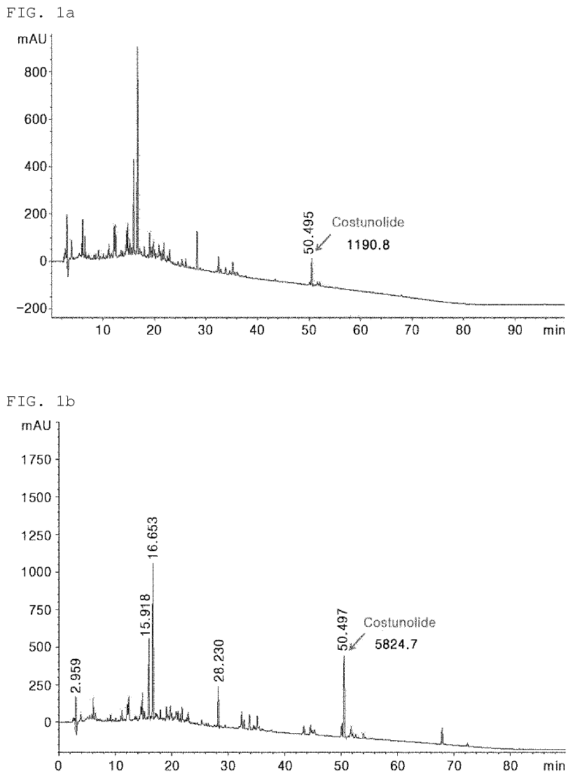

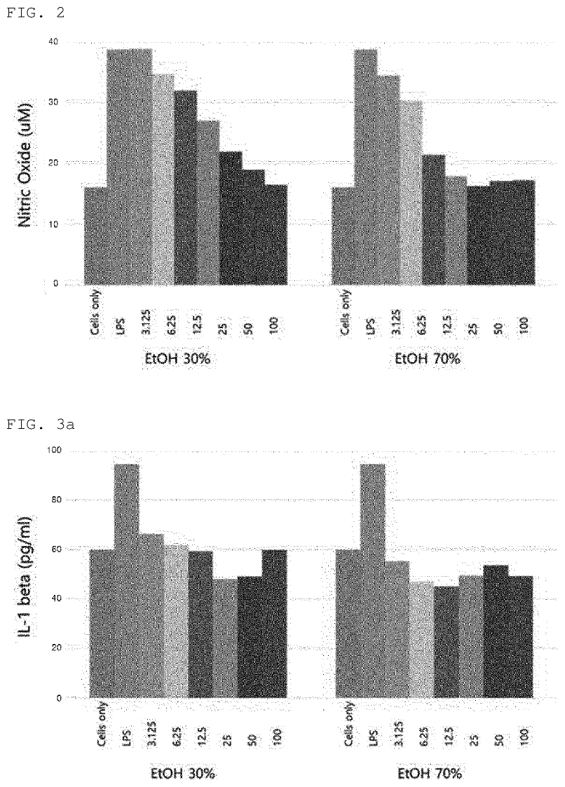

Each of the bay leaf extracts was dissolved at 10 mg/ml and subjected to HPLC to obtain chromatograms (FIG. 1). Each peak in the chromatograms was applied to a calibration curve to calculate concentrations of the ingredients. As a result, the 70% ethanol extract (FIG. 1b) was measured to contain costunolide about 4.9-fold greater than the 30% ethanol extract (FIG. 1a).

<3. Assay for Anti-Inflammatory Activity of Bay Leaf Extract>

1) Culture of Macrophage

Macrophage RAW 264.7 cells used in this experiment were purchased from the American Type Culture Collection (ATCC). The cells were cultured in DMEM (high glucose) supplemented with 10% fetal bovine serum (FBS) and 1% penicillin-streptomycin in a CO2 incubator (Formascientific, Inc.). Under a microscope, differentiation of the cells was identified before use in the experiment.

2) Assay for Anti-Inflammatory Activity in Terms of Nitric Oxide (NO) Level

Activation of the macrophages RAW 264.7 cells was induced by treatment with 200 ng/ml lipopolysaccharide (LPS). The cells were incubated with various concentrations (μg/ml) of the ethanol 30% extract of bay leaves (hereinafter referred to as “EtOH 30%”) and the ethanol 70% extract of bay leaves (hereinafter referred to as “EtOH 70%”) at 37° C. for 18 hours in a 5% CO2 incubator. After 18 hours of incubation, 100 μl of the supernatant from each group was transferred to a new plate. Then, each of the supernatants in new plates was added with 100 μl of a mixture of 1:1 of Greiss reagent A (2% sulfanilamide in 5% phosphoric acid) and Greiss reagent B (0.2% naphthylethylenediamine dihydrochoride), followed by reading absorbance at 540 nm.

Compared to the control group of non-treated cells, the LPS-treated group was measured to have an increased NO level. The bay leaf extracts both significantly decreased levels of NO in a dose-dependent manner (FIG. 2). From the results, it was observed that the bay leaf extracts have anti-inflammatory effects of suppressing excessive inflammatory reactions to prevent tissue injuries.

3) Assay for Anti-Inflammatory Activity in Terms of Inflammatory Cytokine Level

RAW 264.7 cells were incubated with 200 ng/ml LPS alone and in combination with various concentrations (μg/ml) of the bay leaf extracts (EtOH 30% and EtOH 70%) at 37° C. for 18 hours in a 5% CO2 incubator. After 18 hours, the supernatant from each group was measured for levels of inflammatory cytokines IL-1β, IL-6, and TNF-α, using respective ELISA analysis kits (eBioScience 88-7031, 88-7064 USA, BD 555268 USA).

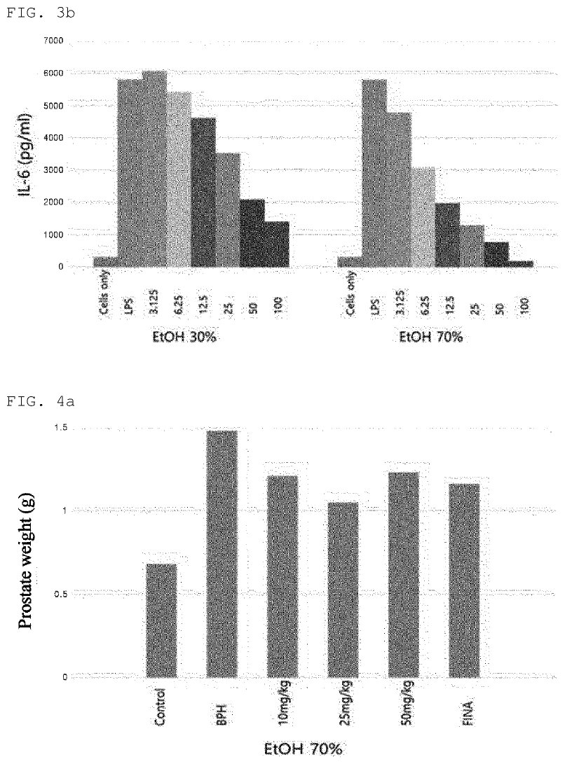

FIG. 3 shows levels of the inflammatory cytokines IL-1β (FIG. 3a) and IL-6 (FIG. 3b) in cultures of LPS-treated macrophages, as measured by ELISA analysis kits. Compared to the cells treated with LPS alone, the cells treated with the bay leaf extracts secreted lower levels of the inflammatory cytokines, demonstrating that the bay leaf extracts have an anti-inflammatory effect.

<4. Assay for Therapeutic Efficacy of Bay Leaf Extract on Benign Prostatic Hyperplasia>

1) Construction of Animal Model of Benign Prostatic Hyperplasia

Male Wistar rats at 8 weeks of age (Samtako, Korea) were acclimated. When the rats weighed about 380 g on average (19 weeks old), they were divided into groups of 7.

After removing the testis therefrom, the rats in one of the groups were allowed to revive for about 24 hours. Propionate testosterone was subcutaneously injected at a dose of 5 mg/kg into the castrated group and the normal group to induce prostate hypertrophy for 8 weeks (BPH-induced groups). After induction of prostate hypertrophy for 8 weeks, the rats were treated as follows. Along with testosterone injection, the bay leaf extracts were orally administered at predetermined doses (mg/kg) once a day (seven times a week) for 8 weeks. Separately, Finasteride, which is a commercially available therapeutic agent for benign prostatic hyperplasia, was orally administered to a positive control.

2) Change in Prostate Weight and Volume

After completion of the experiment, the rats of all the experimental groups were euthanized. The prostate tissues and main organs were excised therefrom and measured for weight and volume using an electronic scale for animal weights and a caliper (mm³) (Table 1).

As a result, the BPH-induced group was observed to significantly increase in prostate weight and volume, compared to the normal control and thus identified for prostate hypertrophy.

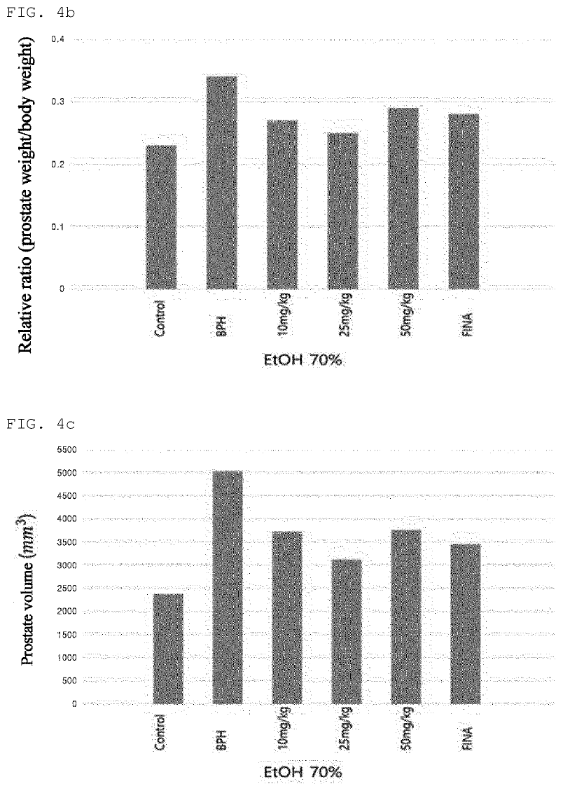

Compared to the BPH-induced group, the bay leaf extract-administered group decreased in prostate weight (FIG. 4a), relative prostate ratio (FIG. 4b), and prostate volume (FIG. 4c), with the similar extent to the BPH drug Finasteride-administered group (FINA). Among others, a dose of 25 mg/kg of the bay leaf extract exhibited higher decreasing effects, compared to the FINA group.

TABLE 1
(weight: g, ratio: %, volume: mm³)

| | | | Prostate | | | | |
|---|---|---|---|---|---|---|---|
| Substance | Test | Weight | weight | Ratio | Prostate | Liver | Kidney |
| administered | group | (a) | (b) | (b/a*100) | volume | weight | weight |
| Non | Control | 291.9 | 0.68 | 0.23 | 2374.36 | 8.84 | 0.66 |
| Non | BPH | 438.2 | 1.48 | 0.34 | 5034.18 | 10.41 | 0.72 |
| EtOH 70% | 10 mg/kg | 444.4 | 1.21 | 0.27 | 3726.09 | 10.37 | 0.67 |
| | 25 mg/kg | 416.7 | 1.05 | 0.25 | 3124.20 | 10.67 | 0.71 |
| | 50 mg/kg | 423.5 | 1.23 | 0.29 | 3761.28 | 10.36 | 0.66 |
| Finasteride | FINA | 426.6 | 1.16 | 0.28 | 3459.59 | 10.36 | 0.65 |
| | (0.8 mg/kg) | | | | | | |

3) Assay for Hepatoxicity and Renal Function

For use in biochemical index analysis, blood samples were taken from the rats of all the groups through abdominal veins. The blood sample was coagulated for about 30 min and centrifuged at 10,000 rpm for 5 min to separate sera. The sera thus obtained were measured for liver function index (AST and ALT), lipoprotein levels (total cholesterol (T-CHO), HDL cholesterol (HDL-C), LDL cholesterol (LDL-C)), and kidney function index (creatine), using a biochemical analyzer (AU480, Beckman Coulter, USA) (Table 2). As a result, statistically significant changes were observed in none of the extract-administered groups, demonstrating that the extracts are free of hepatotoxicity and nephrotoxicity.

TABLE 2

| Substance | | AST | ALT | T-CHO | HDL | LDL | CRE |
|---|---|---|---|---|---|---|---|
| administered | Test group | (U/L) | (U/L) | (mg/dL) | (mg/dL) | (mg/dL) | (mg/dL) |
| Non | Control | 75.9 | 34.7 | 61.4 | 37.9 | 16.9 | 0.2 |
| Non | BPH | 90.7 | 65.9 | 76.0 | 43.9 | 15.9 | 0.3 |
| EtOH 70% | 10 mg/kg | 84.1 | 63.3 | 70.0 | 41.1 | 14.1 | 0.3 |
| | 25 mg/kg | 103.1 | 70.1 | 76.6 | 46.6 | 16.7 | 0.3 |
| | 50 mg/kg | 87.3 | 66.7 | 67.6 | 41.3 | 12.3 | 0.3 |
| Finasteride | FINA | 83.1 | 50.3 | 70.7 | 43.6 | 14.1 | 0.3 |
| | (0.8 mg/kg) | | | | | | |

<5. Assay for Prophylactic Effect of Bay Leaf Extract on Benign Prostatic Hyperplasia>

1) Construction of Animal Model of Benign Prostatic Hyperplasia

Male Wistar rats at 8 weeks of age (Samtako, Korea) were acclimated. When the rats weighed about 320 g on average (12 weeks old), they were divided into groups of 5.

After removing the testis therefrom, the rats in one of the groups were allowed to revive for about 24 hours. Propionate testosterone was subcutaneously injected at a dose of 7 mg/kg into the castrated group and the normal group to induce prostate hypertrophy for 8 weeks (BPH-induced groups). After induction of prostate hypertrophy for 8 weeks, the rats were treated as follows. Along with testosterone injection, the bay leaf extracts were orally administered at predetermined doses (mg/kg) once a day (seven times a week) for 8 weeks. Separately, Finasteride, which is a commercially available therapeutic agent for benign prostatic hyperplasia, was orally administered to a positive control.

2) Change in Prostate Weight and Volume

After completion of the experiment, the rats of all the experimental groups were euthanized. The prostate tissues and main organs were excised therefrom and measured for weight and volume using an electronic scale for animal weights and a caliper (mm³) (Table 3).

As a result, the BPH-induced group was observed to significantly increase in prostate weight and volume, compared to the normal control and thus identified for prostate hypertrophy.

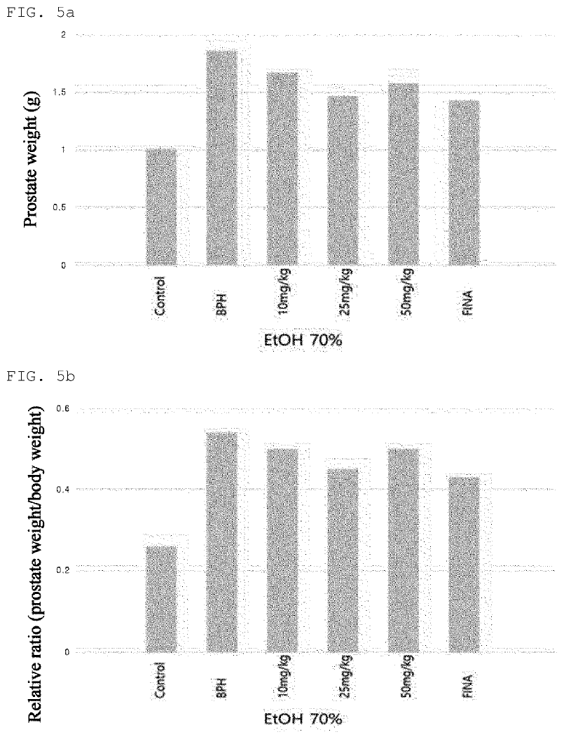

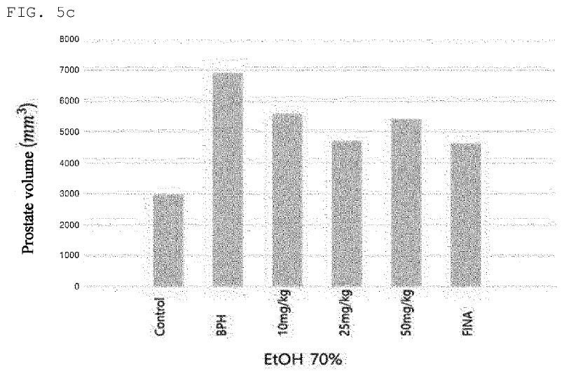

Compared to the BPH-induced group, the bay leaf extract-administered group decreased in prostate weight (FIG. 5a), relative prostate ratio (FIG. 5b), and prostate volume (FIG. 5c). Among others, a dose of 25 mg/kg of the bay leaf extract exhibited higher decreasing effects, compared to the FINA group.

TABLE 3
(weight: g, ratio: %, volume: mm³)

| | | | Prostate | | | | |
|---|---|---|---|---|---|---|---|
| Substance | Test | Weight | weight | Ratio | Prostate | Liver | Kidney |
| administered | group | (a) | (b) | (b/a*100) | volume | weight | weight |
| Non | Control | 393.0 | 1.01 | 0.26 | 2969.39 | 8.12 | 0.65 |
| Non | BPH | 343.2 | 1.86 | 0.54 | 6912.62 | 7.40 | 0.58 |
| EtOH 70% | 10 mg/kg | 337.3 | 1.67 | 0.50 | 5598.94 | 8.05 | 0.68 |
| | 25 mg/kg | 324.8 | 1.47 | 0.45 | 4704.21 | 7.08 | 0.61 |
| | 50 mg/kg | 321.7 | 1.58 | 0.50 | 5417.97 | 7.23 | 0.59 |
| Finasteride | FINA | 328.3 | 1.43 | 0.43 | 4626.31 | 8.09 | 0.58 |
| | (0.8 mg/kg) | | | | | | |

3) Assay for Hepatoxicity and Renal Function

For use in biochemical index analysis, blood samples were taken from the rats of all the groups through abdominal veins. The blood sample was coagulated for about 30 min and centrifuged at 10,000 rpm for 5 min to separate sera. The sera thus obtained were measured for liver function index (AST and ALT), lipoprotein levels (total cholesterol (T-CHO), HDL cholesterol (HDL-C), LDL cholesterol (LDL-C)), and kidney function index (creatine), using a biochemical analyzer (AU480, Beckman Coulter, USA) (Table 4). As a result, statistically significant changes were observed in none of the extract-administered groups, demonstrating that the extracts are free of hepatotoxicity and nephrotoxicity.

TABLE 4

| Substance | | AST | ALT | T-CHO | HDL | LDL | CRE |
|---|---|---|---|---|---|---|---|
| administered | Test group | (U/L) | (U/L) | (mg/dL) | (mg/dL) | (mg/dL) | (mg/dL) |
| Non | Control | 101.0 | 40.4 | 75.6 | 54.0 | 15.2 | 0.3 |
| Non | BPH | 118.0 | 47.8 | 67.6 | 48.2 | 18.2 | 0.2 |
| EtOH 70% | 10 mg/kg | 112.6 | 48.0 | 71.2 | 48.8 | 18.4 | 0.2 |
| | 25 mg/kg | 138.4 | 46.0 | 65.2 | 46.8 | 18.8 | 0.2 |
| | 50 mg/kg | 117.6 | 39.4 | 68.0 | 48.6 | 17.8 | 0.2 |
| Finasteride | FINA | 140.4 | 51.6 | 60.6 | 43.3 | 17.0 | 0.2 |
| | (0.8 mg/kg | | | | | | |

Taken together, the data obtained above indicate that the bay leaf extract has a therapeutic and prophylactic effect on benign prostatic hyperplasia as it increases immunity-related, anti-inflammatory reactions and decreases prostate weight and volume.

While this disclosure includes specific examples, it will be apparent after an understanding of the disclosure of this disclosure that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in described compositions are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A pharmaceutical composition comprising an effective amount of a bay leaf extract as an active ingredient for treatment and prevention of benign prostatic hyperplasia.

2. A health functional food comprising an effective amount of a bay leaf extract as an active ingredient for treatment and prevention of benign prostatic hyperplasia.

3. A method of treating benign prostatic hyperplasia comprising:
preparing a pharmaceutical composition comprising an effective amount of a bay leaf extract an active ingredient; and
administering the pharmaceutical composition to a subject in need of treating the benign prostatic hyperplasia.

4. The method of claim 3, wherein the preparing the pharmaceutical composition comprises extracting the bay leaf extract from a bay leaf using an extraction solvent.

5. The method of claim 4, wherein the extraction solvent comprises an ethanol.

6. The method of claim 5, wherein the ethanol is comprised in the extraction solvent in an amount of 30 vol % to 70 vol % based on a total weight of the extraction solvent.

7. The method of claim 3, wherein the preparing the pharmaceutical composition further comprises:
filtering the bay leaf extract extracted using the extraction solvent; and
lyophilizing and powdering the bay leaf extract into a powder.

8. The method of claim 7, wherein the powder is dissolved in a solvent to prepare the pharmaceutical composition in a solution.

9. The method of claim 8, wherein the solvent is dimethyl sulfoxide, distilled water, or a mixture thereof.

10. The method of claim 8, wherein the solution is administered to the subject in an amount of 10 mg/kg to 50 mg/kg based on a body weight of the subject.
